(19) United States
(12) Patent Application Publication
(10) Pub. No.: US 2004/0167087 A1
(43) Pub. Date: Aug. 26, 2004

(54) Complexation of rna, especially ribozymes, with polyrthylenimines for the stabilization and cellular introduction thereof

(76) Inventors: Achim Aigner, Offenbach (DE); Frank Czubayko, Kirchhain (DE); Thomas Kissel, Satufen (DE); Dagmar Fischer, Meeder (DE)

Correspondence Address: CARELLA, BYRNE, BAIN, GILFILLAN, CECCHI,, STEWART & OLSTEIN, 5 BECKER FARM ROAD, ROSELAND, NJ, 07068, US

(21) Appl. No.: 10/168,125

(22) PCT Filed: Dec. 8, 2000
(86) PCT No.: PCT/EP00/12413

(30) Foreign Application Priority Data
Dec. 14, 1999 (DE) 199 60 206.9

(51) Int. Cl. A61K 48/00

(52) U.S. Cl. 514/44

(57) ABSTRACT

The invention relates to the complexation of RNA with optionally modified polyethylenimines for extracellular and intracellularin vitro and in vivo stabiliztion thereof and for cellular introduction of the complexed RNA in cells. The invention also relates to chemically modified or non-modified, natural or synthetic ribozymes complexes and polyethylenimine (PEI) having any chain length, molar mass and degree of branching, which is chemically modified or non-chemically modified. The invention further relates to the production of complexes and to the use thereof in the protection of RNA against enzymatic and non-enzymatic degradation and their reception in vitro or in vivo in cells. The biological activity of the ribozymes remains intact.

The invention concerns extra- and intracellular stabilisation of modified and unmodified RNA molecules, in particular ribozymes, in relation to enzymatic and non-enzymatic decomposition by complexing with macromolecules based on polyethylene imine (PEI) and the incorporation of those complexes into cells with subsequent liberation of biologically active RNA molecules.

WO-A-96/02655 describes complexes of deoxyribonucleic acids (DNA) with polyethylene imines with a mean molecular weight of 50 kDa and 800 kDa respectively and the use thereof in gene therapy.

WO-A-98/59064 describes complexes of DNA with modified polyethylene imines with a mean molecular weight of 800 kDa and the use thereof for gene transfer in mammal cells.

M A Reynolds (Exogenous delivery of ribozymes, in: Scanlon, K J and Kashani-Sabet, M (eds) Ribozymes in the Gene Therapy of Cancer, pages 41-60, R G Landes Company, 1998) refers to the unpublished observation that, when using fluorescent dye-marked ribozymes (not described in greater detail) in cell culture with a PEI which is not described in greater detail, it was possible to achieve a more than 70% nuclear fluorescence marking of cells.

Ribonucleic acids perform many functions in the cell and are appropriately designated inter alia as messenger RNA (mRNA), transfer RNA (tRNA) and ribosomal RNA (rRNA).

Certain specialised RNA molecules, the so-called ribozymes, are of particular biomedical significance. Ribozymes are catalytically active, small RNA molecules which are capable of cleaving in sequence-specific manner other RNA molecules which are significant for essential cell functions. That therefore results in down-regulation of the expression of the respective gene, that is to say inhibition of specific gene functions. Various ribozymes or ribozyme classes can be distinguished according to their structure, function, activity and size (see Table 1). Possibly associated protein components serve in that respect for stabilisation and efficiency enhancement and are not the actual support of catalytic activity.

TABLE 1
Summary of the various ribozymes with their activity,
function and size.

| Ribozymes | Activity | Function | Size |
|---|---|---|---|
| Hammerhead | Ribonuclease (5′OH) | Replication | ≈35 nucleotides |
| motif | | | |
| Hairpin motif | Ribonuclease (5′OH) | Replication | ≈60 nucleotides |
| HDV ribozyme | Ribonuclease (5′OH) | Replication | ≈80 nucleotides |
| RNase P | Ribonuclease (5′P) | pre-tRNA | >275 nucleotides |
| | | cleaving | |
| Intron group 1 | Ester interchange | Splicing | >200 nucleotides |
| Intron group 2 | Ester interchange | Splicing | >600 nucleotides |

Ribonuclease (5′OH) stands for a ribonuclease activity which produces a 5′OH and a 2′-3′-cyclic phosphate residue, ribonuclease (5′P) stands for a reaction in which there is a 5′-phosphate and a 3′-OH termination.

The hammerhead ribozymes belong to the smallest and best investigated catalytic RNA molecules and are capable of sequence-specifically cleaving other RNA molecules. The most frequently used hammerhead ribozyme is derived from the original ribozyme in accordance with Haseloff-Gerlach (Natur, 334, 585-59 (1988)) which was modified and reduced to a catalytic center including 22 nucleotides. That nuclear sequence whose three-dimensional and catalytically active structure is formed by Watson-Crick base pairing is flanked by two arms which also on the basis of Watson-Crick base pairing hybridise specifically to the complementary sequence portion of the target RNA and thus bring the catalytically active region of the ribozyme into sufficient spatial proximity with the cleavage location of the target RNA. Specific cleavage with subsequent cellular decomposition of RNA molecules of given genes makes it possible in that way to use ribozymes inter alia for the diagnostic and therapeutic inhibition of specific gene functions. The greatest obstacles which hitherto had to be overcome for the use of ribozymes are the very high level of susceptibility of the molecules in relation to enzymatic decomposition by virtue of ubiquitously present intra- and extracellular RNA-decomposing enzymes (ribonucleases, RNases) and poor penetration of the ribozymes in cells.

While intracellularly produced, natural RNA molecules (for example mRNA and tRNA) in the intact cell due to structure-specific properties and cellular modifications under given conditions have at least a limited stability, synthetic RNA molecules such as ribozymes can only be protected from that rapid decomposition by chemical modifications. Those chemical modifications however suffer from major disadvantages:

functional limitation to complete loss of activity of the ribozyme

formation of modified ribozymes with detrimental and in particular cell-toxic properties

a very high level of complication and expenditure for the development of those chemical modifications and the chemical conversion thereof.

Nonetheless, in consideration of the potentially outstanding therapeutic significance of ribozymes, numerous endeavours have been undertaken for stabilisation by chemical modifications (for example Heidenreich et al, J Biol Chem 269, 2131-2138 (1994); Pieken et al, Science 253, 314-317 (1991)). It will be noted that here too those modifications firstly generally result in inactivation of the ribozyme and secondly require very complicated and expensive synthesis.

To sum up it can be said that an effective method which

protects RNA molecules from degradation but

alters the structure of the molecules as little as possible and

as far as possible does not influence their function is of great fundamentals- and use-oriented scientific and economic interest.

The present invention describes that RNA molecules, in particular ribozymes, can be protected extra- and intracellularly from enzymatic and non-enzymatic decomposition by complexing with macromolecules based on polyethylene imine (PEI). Even ribozymes which otherwise are subject to particularly rapid degradation are in that case completely protected from decomposition without a loss of activity occurring. The PEI-ribozyme complexes are stable even in biological, serum-bearing media. In addition there is afforded a complex with a low degree of toxicity, which is in a position to very efficiently incorporate ribozymes in functionally active form into living cells and thus manipulate cellular processes in the desired form. Going beyond ribozymes the complexing of RNA finally permits much more extensive, varied use for the stabilisation of the most widely varying RNA molecules.

Thus in summarised form the method is suitable for:

stabilisation of RNA molecules, in particular ribozymes, in relation to enzymatic or non-enzymatic decomposition in vitro and in vivo, extracellularly or intracellularly,

stabilisation in terms of the storage of RNA molecules, in particular ribozymes,

stabilisation in terms of transportation of RNA molecules, in particular ribozymes,

stabilisation of RNA molecules, in particular ribozymes, in terms of the use thereof in laboratory experiments,

as a vector for RNA molecules, in particular for ribozymes in gene transfer applications in vivo (gene therapy), and

as a vector for RNA molecules, in particular for ribozymes, for use in cell culture experiments.

The invention therefore concerns complexes of RNA, in particular ribozymes, and polyethylene imine (PEI), in which the PEI has a molecular weight of between 700 and 1,000,000 Daltons (Da), preferably between 1400 and 25,000 Da, in particular between 1600 and 15,000 Da. Preferred complexes in accordance with the invention are those in which the molar ratio of the nitrogen atoms in the PEI to the phosphorus atoms in the RNA (N/P value) is between 1 and 100, preferably between 2 and 40, in particular between 3 and 10.

In the complexes according to the invention PEI can be modified by cellular ligands. That modification can be effected prior to or after complexing of the RNA. Likewise modification can be implemented by hydrophilic polymers such as for example polyethylene glycol (PEG) of a molecular weight of between 100 and 10,000,000 g/mol, preferably between 1000 and 100,000 g/mol and in particular between 5000 and 50,000 g/mol. In that respect a PEI can be modified by one or more hydrophilic polymers or a hydrophilic polymer by one or more PEIs. Such modified PEIs and methods of producing same are described in international application PCT/EP00/06214; by reference thereto the content thereof is made part of the disclosure of the present application.

The RNA to be protected can be any RNA, in particular a messenger RNA (mRNA), a ribosomal RNA (rRNA), a transfer RNA tRNA), a nuclear RNA, a RNA-ribozyme, an in vitro transcribed RNA or a chemically synthesised RNA, in which respect all said RNA molecules can be chemically modified. In that respect the RNA may have between 10 and 10,000 nucleotides, preferably between 15 and 1500 nucleotides, in particular between 20 and 1000 nucleotides. In the case of ribozymes chain lengths of 20-600 nucleotides, in particular 35-80 nucleotides are preferred.

Preferred complexes are those in which the RNA is a ribozyme, in particular a hammerhead or hairpin ribozyme, or which involves RNA aptamers or antisense sequences. In addition, preferred complexes are ones in which the RNA entirely or in part codes for a therapeutically effective peptide or protein or however for a cellular suicide gene.

The invention further concerns a method of producing a complex according to the invention in which the RNA is complexed with the PEI possibly modified by cellular ligands or by further polymers, by mixing of the dilute solutions.

Production of the complexes according to the invention is technically executed as follows:

For complexing of RNA, in particular ribozymes, with PEI, an aqueous solution of the RNA is produced. The RNA can be chemically modified or unmodified. Methods according to the invention are preferred, in which the RNA concentration is preferably between 0.1 and 100 μg/ml, in particular between 10 and 40 μg/ml.

Suitable complexing reagents are commercial preparations of modified or unmodified PEIs as well as non-commercial modified or unmodified PEIs such as for example LMW-PEI (Fischer et al Pharm Res 16, 1273-9 (1999)). To produce the respective stock solution the PEI in question is dissolved in bi-distilled water and the pH-value is set with an approximately 100 mM HCl solution to a neutral pH-value of preferably between 7 and 8, particularly preferably pH 7.4.

Immediately prior to implementing the complexing reaction the PEI stock solution (in regard to the amount used, see Tables 2 and 3 hereinafter) is set with an aqueous approximately 150 mM neutral NaCl solution, pH between 7 and 8, preferably 7.4, to the desired volume and incubated for some time, preferably at least 10 minutes, at ambient temperature.

The RNA (in regard to the amount used see for example Tables 2 and 3 hereinafter) is adjusted with an aqueous approximately 150 mM NaCl solution, pH between 7 and 8, preferably 7.4, to the desired volume, particularly preferably approximately the same volume as that of the PEI solution, and incubated for some time, preferably at least 10 minutes at ambient temperature.

Then the PEI and the RNA solutions (mixing ratios see for example Tables 2 and 3 hereinafter) are mixed and incubated for some time, preferably at least 10 minutes at ambient temperature. The mixture can be shaken or vortexed one or more times.

The complex composition and the net loading are calculated on the basis of RNA phosphorus to PEI nitrogen and specified as the nitrogen-phosphorus ratio (N/P ratio, see above), in accordance with Boussif et al (Proc Natl Acad Sci 92, 7297-7301 (1995)) or the phosphorus-nitrogen ratio.

The following formula serves as the basis for calculation:

1 μg RNA=3 nmol phosphorus and

2 μl PEI-sol.=10 nmol nitrogen.

The composition ratios of a standard composition with 2 μg RNA and the corresponding amounts of PEI are summarised in the following Tables by the example of two PEI preparations (however see also more extensive information hereinafter):

TABLE 2
Overview of HMW-PEI composition ratios which
can be used.

| Composition ratios | Volumes | |
|---|---|---|
| RNA/HMW-PEI | HMW-PEI stock | HMW-PEI |
| (equivalent) | solution 0.9 mg/ml [μl] | absolute [μg] |
| 1 + 1 | 6 | 0.27 |
| | (1:10 diluted) | |
| 1 + 6.67 | 4 | 2.7 |
| 1 + 10 | 6 | 1.8 |
| 1 + 13.33 | 8 | 3.6 |
| 1 + 20 | 12 | 5.4 |

TABLE 3
Overview of LMW-PEI composition ratios which can
be used.

| Composition ratios | Volumes | |
|---|---|---|
| RNA/LMW-PEI | LMW-PEI stock | LMW-PEI |
| (equivalent) | solution 0.9 mg/ml [μl] | absolute [μg] |
| 1 + 1 | 6 | 0.54 |
| | (1:10 diluted) | |
| 1 + 2 | 12 | 1.08 |
| | (1:10 diluted) | |
| 1 + 13.33 | 8 | 7.2 |
| 1 + 20 | 12 | 10.8 |
| 1 + 26.67 | 16 | 14.4 |
| 1 + 40 | 24 | 21.6 |
| 1 + 66.66 | 40 | 36 |
| 1 + 100 | 60 | 54 |

After incubation has been effected the complexes, being ready for use, can be used or can be introduced into further chemical modifications.

The invention concerns the methods set forth for protecting RNA, in particular ribozymes, from enzymatic and non-enzymatic decomposition, in which the RNA molecules are stabilised in the complex with modified or unmodified PEI, and methods in which RNA ribozymes are protected from decomposition by nucleases.

A further aspect of the invention is methods of incorporating RNA, in particular ribozymes, into cells, preferably into eucaryontic cells, and the intracellular liberation thereof in biologically active form.

The invention further concerns compositions which contain an effective amount of at least one RNA-PEI complex according to the invention, wherein the RNA can be present in any concentration which is sub-toxic for cells or for the respective organism or which is still not permanently cell-damaging at least in relation to treatment over several hours.

The invention further concerns pharmaceutical compositions containing an effective amount of at least one RNA-PEI complex according to the invention and one or more physiologically harmless carriers.

The RNA/PEI ratio can be specified by way of the molar ratio of the nitrogen atoms in the PEI to the phosphorus atoms in the RNA (N/P value). The N/P value may not be too low for complete complexing of the RNA.

The N/P value of the complexes however can fluctuate in particular upwardly over a wide range, it can be in the range of between about 1 and about 100. Preferably the ratio is between about 2 and about 40, particularly preferably the ratio is between 3 and 10.

For the specific situation of use, for example for the respective RNA type, in particular for ribozymes, for PEI of given molar masses or for the respective cell type, it is possible to ascertain optimum N/P values by preliminary tests. In that respect, with a stepwise increase of the ratio under otherwise identical conditions the extent of protection of the RNA, in particular the ribozyme, the efficiency of the RNA, in particular ribozyme, transfer, intracellular activity of the ribozyme and the exclusion of toxic effects of the complex or individual constituents are the most important parameters, possibly also material saving or time kinetics.

The PEI used here for the complexing procedure involves molecular weights of between 700 and 1,000,000 Da. Over the entire molecular weight range and irrespective of the degree of branching, all PEI molecules, modified or unmodified, are suitable for the protection of RNA molecules, in particular ribozymes, but they have differences in regard to cellular reception of the complexes. Large PEI molecules already have optimum efficiency in terms of cellular reception at low N/P ratios, but they are more toxic than small PEI molecules. With the same amount of RNA, in particular ribozyme, the latter require a higher N/P ratio for the complexing operation, without in that respect being comparably toxic. Which PEI molecule is specifically used can be ascertained in preliminary tests.

They can be described for example by structural formula I:

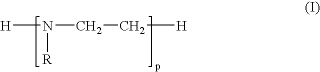

wherein R is a residue of the formula II:

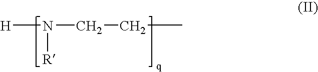

in which R′ represents hydrogen, p is a positive integer, q is 0 or a positive integer and (p+q) is between 15 and 25,000, preferably between 30 and 600, in particular between 35 and 400. In addition II can also be branched. Then R′ is a residue of a PEI which is constructed similarly to formula II.

Preferably in accordance with the invention PEI molecules are in the molecular weight range of between 1400 and 25,000.

LMW-PEI (‘low molecular weight PEI’) is suitable for that purpose, which is produced from aziridine (ethylene imine monomer) by ring-opening polymerisation in aqueous solution with acid catalysis. The synthesis procedure is described in Fischer et al, Pharm Res 16, 1273-9 (1999).

In addition it is possible to use commercially available PEI preparations of different molecular weights. Examples are PEI 700 Da, PEI 2000 Da and PEI 25000 Da (Aldrich) and, from BASF under the marks Lupasol®, 800 Da, (Lupasol®FG), 1300 Da (Lupasol®G 20 water-free), 1300 Da (Lupasol® G 20), 2000 Da (Lupasol® G 35) and 2500 Da (Lupasol® WF).

The PEI can be modified by a cellular ligand, identified hereinafter by ‘Q’. The term cellular ligand is used to denote a group which exerts a specific or non-specific biological function, in particular representing a binding partner for interactions with receptors or other cellular proteins. The modification with a ligand serves in particular for the target cell-specific reception of an RNA-PEI-Q complex in higher eucaryontic cells and the cell nucleus thereof, wherein the RNA is preferably a ribozyme (gene targeting).

Q can thus also be a ligand for a specific interaction and for the preferred reception of the RNA-PEI-Q complex in target cells, tissue or organs. Examples of Q are proteins, in particular:

antibodies or antibody fragments such as Fab, F(ab₂), scFv, or

cyto- or lymphokines such as interleukins (IL-2 to X), interferon GM-CSF, or

growth factors such as EGF, PDGF, FGF, EPO, or

integrins such as ICAM, VCAM, or

glycoproteins such as lectins or glycosylated proteins (see above, or

lipoproteins such as LDL, HDL, or

transporter proteins such as transferrin, or

peptides such as LH-RH, calcitonin, oxytocin, insulin, somatostatin, IGF, RGD, or

carboyhydrates such as galactose, mannose, glucose, lactose, or

hormones such as steroids, THR, or

vitamins such as vitamin B12 or folic acid.

For certain in vivo applications it is required in regard to a high level of gene transfer efficiency that the complexes according to the invention are present in a high concentration. The complexes according to the invention have the advantage that they can be brought to the required high concentration from dilute solutions for example by way of physical methods such as ultra-centrifuging, ultra-filtration or pressure analysis, without the occurrence of any aggregate formation worth mentioning or other alteration in the complexes, which would adversely affect the level of gene transfer efficiency.

In particular the composition is present in the form of a pharmaceutical composition. In that configuration the composition serves not only to protect RNA but also for the transfer of the RNA into cells, in particular eucaryontic cells, particularly preferably mammal cells, in vivo or in vitro. It contains as its active constituent a complex which includes a therapeutically effective RNA, in particular a ribozyme or a plurality thereof. By means of the pharmaceutical composition according to the invention, in relation to local application, it is possible to achieve a high level of concentration of therapeutically effective RNA in the target cells or in in vivo experiments in the target tissue. In terms of systemic application the composition has the advantage that the complexes are not subject to either non-specific binding or immune system-induced decomposition, because of the prevention of opsonisation.

Due to the reduction in or prevention of non-specific bindings with the simultaneous introduction of (cell type-specific) cell binding ligands into the complexes, it is possible to achieve specific targeting in relation to given cells, organs and tissues and thus target-directed gene expression (for example in tumor tissue), or another effect after systemic administration.

In tissues with increased vessel permeability or with vessel damage, the RNA-PEI complexes according to the invention can issue from the bloodstream into the surrounding tissue and accumulate there. Regions where such ‘passive targeting’ occurs to an enhanced degree are for example inflammation regions and tumors with a good supply of blood.

In many of those cases a sufficiently high level of target selectivity is then already achieved so that the linking of PEI to target cell-specific ligands can be omitted.

The pharmaceutical composition can be used inter alia to advantage for the therapy of tumor diseases in order to administer RNA, in particular ribozymes, intratumorally or systemically.

A further application in which the advantages of the composition according to the invention apply is so-called genetic tumor vaccination. The complexes used in that respect contain RNA, coding for one or more tumor antigens or fragments thereof.

The pharmaceutical composition according to the invention of the complex is preferably in the form of lyophilisate, possibly with the addition of sugar such as saccharose or dextrose in an amount which gives a physiological concentration in the solution when ready for use. The composition according to the invention can also be deep-frozen (cryopreserved) or can be in the form of a cooled solution and contain further additives.

The compositions according to the invention can possibly be in the form of a kit, wherein the individual components RNA, in particular ribozymes, on the one hand, and PEI to which a ligand is possibly coupled, on the other hand, are present in separate containers.

The following Examples serve to illustrate the invention without the invention being restricted thereto.

### EXAMPLE 1

Protection of unmodified, chemically synthesised RNA oligonucleotides (37 nucleotides) from enzymatic decomposition by complexing with PEI.

The complexing as between hammerhead ribozymes and PEI, which has hitherto not been described in the literature, was investigated in relation to the stability of the ribozyme in relation to degradation by serum nucleases. A prolongation achieved in that way in the half-life in the presence of serum would permit systemic application of unmodified ribozymes which are superior to their chemically modified analogs in relation to catalytic activity and production costs.

Two polyethylene imine solutions of different molecular weights were used for the experiments. HMW-PEI was obtained in the form of a 50% aqueous solution from the company Fluka (Neu-Ulm) of a molecular weight of 600-1000 kDa (manufacturer specification, determined viscosimetrically). To produce the stock solution 9.0 mg of HMW-PEI was dissolved in 8 ml of bi-distilled water, the pH-value was set to 7.4 with 0.1 N HCl solution and it was made up to 10.0 ml with bi-distilled water.

LMW-PEI was produced from aziridine (ethylene imine monomer) by ring-opening polymerisation in aqueous solution with acid catalysis. The molecular weight of≦10 kA was determined by means of laser scatter light measurement by the company Wellensieck (Minden) (Fischer et al, Pharm Res 16, 1273-9 (1999)). To produce the stock solution 9.0 mg of HMW-PEI was dissolved in 8 ml of bi-distilled water, the pH-value was set to 7.4 with 0.1 N HCl solution and it was made up to 10.0 ml with bi-distilled water.

The RNA oligonucleotide involves a hammerhead ribozyme of a length of 37 bases which was provided with a 5′-O-DMT-ON-2′-Ofpmp protective group (MWG-Biotech GmbH, Ebersberg). For the stability investigations the protective group was split off by means of a protective group removal kit that was also supplied (Cruachem, Glasgow) by acid hydrolysis and the RNA from which protection had been removed obtained by ethanol precipitation.

Complexing of the oligonucleotides with PEI was implemented in accordance with the above-specified instructions.

Five different oligonucleotide-PEI ratios were selected for HMW-PEI and seven such ratios for LMW-PEI. The choice was made based on complexing tests as between HMW- and LMW-PEI and the cMV-nlacZ-plasmid (Fischer, Nichtvirale Vektoren zur Gentherapie: Kationische Polymere als Vektorsysteme für den Transfer von DNA Cuvillier Verlag, Göttingen, 1st edition (1998)).

The composition ratios of a standard composition with 2 μg of oligo and the amounts of PEI reacted therewith are summarised in the following Tables:

TABLE 4
Overview of HMW-PEI composition ratios used.

| Composition ratios | Volumes | |
|---|---|---|
| RNA/HMW-PEI | HMW-PEI stock | HMW-PEI |
| (N/P ratio) | solution 0.9 mg/ml [μl] | absolute [μg] |
| 1 + 1 | 6 | 0.27 |
| | (1:10 diluted) | |
| 1 + 6.67 | 4 | 2.7 |
| 1 + 10 | 6 | 1.8 |
| 1 + 13.33 | 8 | 3.6 |
| 1 + 20 | 12 | 5.4 |

TABLE 5
Overview of LMW-PEI composition ratios used.

| Composition ratios | Volumes | |
|---|---|---|
| RNA/LMW-PEI | LMW-PEI stock | LMW-PEI |
| (N/P ratio) | solution 0.9 mg/ml [μl] | absolute [μg] |
| 1 + 1 | 6 | 0.54 |
| | (1:10 diluted) | |
| 1 + 2 | 12 | 1.08 |
| | (1:10 diluted) | |
| 1 + 13.33 | 8 | 7.2 |
| 1 + 20 | 12 | 10.8 |
| 1 + 26.67 | 16 | 14.4 |
| 1 + 40 | 24 | 21.6 |
| 1 + 66.66 | 40 | 36 |

Incubation of ribozyme-PEI complexes with fetal calf serum (FCS). For initial orientation as to whether PEI protects ribozymes from decomposition by serum nucleases the complexes HMW-PEI 1+13.3 and LMW-PEI 1+66.6 were selected and incubated over 5 and 15 minutes at 37° C. in 100% FCS. The choice of those two complexes was made on the basis of the described high transfection rates of plasmid complexes in comparison with the other HMW- and LMW-PEI complexes (Fischer et al, Pharm Res 16, 1273-9 (1999)). After incubation the serum proteins were denatured by means of heat inactivation by the batches being incubated for 30 minutes in a water bath at 70° C. The addition of 8 μl of 10% SDS solution caused the destruction of still active enzymes and at the same time the ribozymes were liberated from the complexes with PEI. The mixture was then separated by a gel-electrophoretic procedure and intact RNA bands were detected by means of a fluorescing cyanin dye (SYBR-Gold®, MoBiTech, Göttingen) which binds to single-strand or double-strand DNA or RNA.

In contrast to the non-complexed ribozymes which were already completely degraded after 5 minutes the ribozymes complexed with HMW-PEI or LMW-PEI were almost completely protected from decomposition by the nucleases contained in the serum over a 15 minute incubation duration.

HMW-PEI and LMW-PEI complexes were now produced in the previously investigated nitrogen-phosphate ratios and incubated for 15-120 minutes in 10% serum similarly to the preceding experiment. To check the experiment results, besides the non-complexed ribozymes, ribozymes in the complexes HMW-PEI (1+1) and LMW-PEI (1+2) were also applied, in which, as is known from preliminary tests, complete complexing does not occur and which consequently should not afford protection for the RNA from degradation. Both the non-complexed ribozymes and also the RNA oligonucleotides which were complexed in excessively low nitrogen-phosphate ratios with PEI were, as expected, completely degraded by the serum nucleases both after 60 minutes and also after 120 minutes. The other two complexes (HMW-PEI 1+13.3 and LMW-PEI 1+66.6) which, by virtue of complete complexing of the ribozyme by PEI, had already exhibited a protective action in relation to enzymatic decomposition over a period of 30 minutes, stabilised the ribozyme even over 60 minutes and 120 minutes.

Finally it can thus be noted that HMW-PEI and LMW-PEI are in a position to protect complex RNA ribozymes, as from nitrogen-phosphate ratios of 1+6.67 (HMW-PEI) and 1+13.3 (LMW-PEI) from degradation by serum nucleases over a period of at least 120 minutes. That prolongation of the half-life from a few minutes to more than 2 hours makes it possible to use unmodified RNA ribozymes both for in vitro and also in vivo transfection experiments.

### EXAMPLE 2

Protection of unmodified, in vitro transcribed RNA molecules (300-800 nucleotides) from enzymatic decomposition by complexing with PEI.

As Example 1 shows, it was possible for shorter RNA molecules which in their length correspond to hammerhead ribozymes or tRNA molecules to be stabilised by PEI complexing. A second step involved investigating whether longer RNA molecules (up to 1000 nucleotides) which correspond to mRNA molecules are also protected in the same way. As such long RNA molecules cannot be chemically synthesised, here the method of in vitro transcription was used (in vitro transcription kit from Promega). In this case a DNA template (plasmid DNA) is transcribed by means of RNA polymerase in vitro into RNA. That RNA is similar in respect of its length and structure to cellular rRNA although important stabilising elements such as the 5′-Cap structure and the 3′-poly-A-Tail are missing. Accordingly in vitro transcribed RNA is more sensitive in relation to decomposition by ribonucleases than natural cellular mRNA. For better representation and quantification the RNA was radioactively marked by the incorporation of ³²P-marked nucleotides. For the experiments, two different RNA molecules were produced, which were respectively 300 and 800 nucleotides long.

The radioactively marked RNA molecules, complexed with LMW-PEI vs. non-complexed, were incubated either with recombinant RNase A (final concentration 20 ng/ml) or fetal calf serum (1% -40% final concentration) for various periods of time (0-120 minutes). After incubation the RNA was electrophoretically separated on a denaturing formaldehyde agarose gel and the RNA was then transferred to a RNA-binding nylon membrane by means of the Northern blotting technique. For qualitative evaluation X-ray films were exposed on the dried membrane for 2-8 days, in part using an amplification system (BioMax). For quantification purposes the radioactive signals on the nylon membrane were detected by way of a Phospoimager from Molecular Dynamics and evaluated by means of analysis software from the same company.

The results are summarised in FIG. 1. While as expected the non-complexed RNA is almost completely decomposed after just 15 minutes, after incubation with RNase A (final concentration 20 ng/ml), more than 60% of the molecules are still intact even after 2 hours, out of the PEI-complexed RNA. A very similar kinetic also occurs after incubation with fetal calf serum (FIG. 2). Here the non-complexed RNA is completely decomposed after about 30 minutes after incubation with 1% fetal calf serum while the PEI-complexed RNA is still fully intact even after 120 minutes. Even with an increase in the concentration to 40% fetal calf serum the PEI-complexed RNA is markedly more stable in comparison with the non-complexed RNA. The results were identical for the RNA molecules of different lengths. The data of the graphs were averaged from two independent experiments.

To sum up it can be noted that in vitro transcribed RNA molecules (300-800 nucleotides long), non-complexed, were as expected decomposed by ribonucleases within a very short time. By complexing with low-molecular PEI (LMW-PEI) it was possible for the half-life of the RNA in fetal calf serum (up to 40% end concentration) to be prolonged to over two hours.

### EXAMPLE 3

Chemically synthesised, unmodified RNA ribozymes are incorporated by complexing with LMW-PEI into tumor cells and cleave their target RNA with high efficiency.

After the first two Examples demonstrated stabilisation of various RNA molecules by LMW-PEI, investigations were made to ascertain whether RNA complexed with LMW-PEI passes into the cells and is intracellularly unaffected in respect of its biochemical function.

For that purpose the chemically synthesised hammerhead ribozymes described in Example 1 were used. Those ribozymes are directed against the human mRNA of a fibroblast growth factor-binding protein (FGF-BP) (Czubayko et al, Nature Med 3 (10)), 1137-1140 (1997)). The FGF-BP protein is expressed in many human tumor and tumor cell lines derived therefrom. The functionality of the ribozymes could be demonstrated in earlier experiments by virtue of the fact that, after stable transfection of the eucaryontic ribozyme expression vectors in tumor cells (ME-180 cervix carcinoma cell line, LS174T colon carcinoma cell line) the target gene on the m-RNA- and on the protein level was markedly reduced (Czubayko et al, Nature Med 3 (10)), 1137-1140 (1997)).

While this gene transfer method is completely unsuitable for for example reaching solid tumors or metastases in vivo and to provide therapy in respect thereof, LMW-PEI ribozyme complexes could afford an outstanding alternative here. It is known from preliminary data of other groups that LMW-PEI provides highly efficiently and at low toxicity for the cellular reception of deoxyribonucleic acids.

For the experiments described herein, the chemically synthesised ribozymes directed against FGF-BP (1.0 and 0.1 μg; about 10 and 1 nmol respectively final concentration) were used and introduced by means of LMW-PEI into ME-180 tumor cells. The two different ribozyme amounts were moreover complexed in two different ratios with LMW-PEI (P/N ratios of 1:13 and 1:53). Those ribozyme-PEI complexes were given for 4 hours in 10 ml of serum-free IMDM medium (company PAA) to about 1×10⁶ ME-180 cells which grew as a monolayer on 10 cm cell culture dishes. As a control for non-specific effects ME-180 cells were treated with the same amount of LMW-PEI without complexed ribozyme. Thereafter the transfection medium was aspirated and the cells were again put on to normal growth medium (IMDM plus 10% FCS). At different moments in time after commencement of transfection (6-36 hours) the cells were lysed and the entire cellular RNA isolated in accordance with the standard protocol (trizol reagent, from Sigma). The RNA produced in that way was analysed by means of the Northern blotting procedure, in which respect the probe used was an FGF-BP cDNA radioactively marked by means of ³²P. Detection of FGF-BP mRNA was represented by means of autoradiograms and phosphorimagers and quantified. As can be seen from FIGS. 3-5 transfection with LMW-PEI ribozymes resulted in a very great ribozyme-specific reduction in FGF-BP gene expression. The maximum reduction was about 70% compared to the control cells. Both ribozyme-PEI ratios of 1:13 and 1:53 exhibited similar effectiveness. Interestingly the lower ribozyme dose of 0.1 μg was somewhat more effective in both PEI ratios so that it can be assumed that still lower doses can also already be highly efficient.

The pronounced inhibition of the FGF-BP gene expression by a maximum of ⁷⁰% after 24 hours is still more noteworthy if it is viewed in the context of comparative experiments. In transient transfection studies with various gene transfer vectors (for example plasmid DNA, adenoviral vectors, various lipid vesicles) we have not been able to observe inhibition effects of more than 25% either with ribozymes or with DNA oligonucleotides. In particular we have not been able to demonstrate any reduction in the target RNA with chemically stabilised RNA/DNA ribozyme hybrid molecules (these are viewed as the gold standard) which were made available to us by the leading company in this field (Ribozyme Pharmaceuticals, Boulder, Colo., USA) (unpublished data).

It can therefore be emphasised that this combination of LMW-PEI and unmodified RNA ribozymes is a new and extraordinarily effective agent for the selective reduction of the expression of genes according to choice.

### EXAMPLE 4

Chemically synthesised, unmodified RNA ribozymes complexed with LMW-PEI after intraperitoneal application in athymic nude mice result in specific inhibition of growth of human melanoma xenotransplants.

After the first three Examples demonstrated the stabilisation of various RNA molecules by LMW-PEI and the biological activity of ribozymes complexed with LMW-PEI in human tumor cells in cell culture experiments, the question investigated was whether the systemic application of ribozymes complexed with LMW-PEI in athymic nude mice can inhibit the growth of human tumor cell xenotransplants.

For that purpose use was made of chemically synthesised hammerhead ribozymes which are directed against the human mRNA of a heparin-binding growth factor (pleiotrophin=PTN) (Czubayko et al, PNAS 93, 14753-14758 (1996)). The PTN protein was expressed in many human tumors and tumor cell lines derived therefrom. The functionality of the ribozymes against PTN could be demonstrated in earlier experiments insofar as, after stable transfection of the eucaryontic ribozyme expression factors in tumor cells (1205 melanoma cell line) the target gene was markedly reduced on the mRNA and on the protein levels (Czubayko et al, PNAS 93, 14753-14758 (1996)). In addition the growth of 1205 tumor cell xenotransplants in athymic nude mice was markedly reduced in the ribozyme-transfixed cell lines. A reduction in the PTN-protein amount by 25% already resulted in an inhibition of tumor growth in nude mice by about 33%. Those preliminary data make this tumor model particularly suitable for demonstrating the efficiency of new gene targeting methods in vivo as the extent of the ribozyme-afforded reduction in PTN expression can be directly inferred from the inhibition of the tumor growth.

While stable and transient plasmid-DNA gene transfer methods are totally unsuitable for reaching for example solid tumors or metastases in vivo and to provide therapy in respect thereof LMW-PEI ribozyme complexes could afford an outstanding alternative here.

In preliminary tests in respect of cell culture it was initially found that a ribozyme/LMW-PEI ratio of 1:66.66 in 1205 cells gives optimum transfection results. For the animal test a dose of 8 μg of ribozyme per injection per mouse was used. With a total weight of 20 g per mouse that gives a ribozyme dose of 0.4 mg/kg bodyweight. That dose should be applied to the test animals intraperitoneally (i.p.) every two days over three weeks. The ribozyme/LMW-PEI complexes were produced freshly each morning prior to injection in accordance with Example 1.

For the animal testing, in a total of 15 test animals, two 1205 tumor cell xenotransplants were respectively induced in the flanks of the animals by subcutaneous injection of 1×10⁶ tumor cells. After the tumors had grown after three days to a size which can be seen and felt of about 2×2 mm the animals were randomly divided into a treatment group and a control group. In the treatment group 8 animals were injected i.p. at an interval of 2 days with ribozyme/LMW-PEI complexes (N/P ratio=1:66.66; 8 μg of ribozyme/injection). In the control group 7 animals were injected i.p. at the same interval with the same amount of LMW-PEI (72 μg per injection) without ribozyme. After a total of 7 injections three days after the last injection the animals were killed, the subcutaneous tumors were removed and the tumor volume was determined. The control group (7 animals=14 tumors) gave a mean tumor size of 2061±481 mm³ (mean value and standard deviation) while in the treatment group (8 animals=16 tumors) there was a mean tumor size of 1066±195 mm³ (mean value and standard deviation). That specific ribozyme-afforded inhibition of the 1205 tumor xenotransplant growth by 48% was significant in the unpaired t-test (p<0.05).

This pronounced inhibition of the 1205 tumor growth by about 50% after 20 days is even more remarkable if it is viewed in the context of comparative investigations. In similar animal tests with chemically stabilised RNA/DNA ribozyme hybrid molecules (viewed as the gold standard) which were made available to us by the leading company in this field (Ribozyme Pharmaceuticals, Boulder, Colo., USA), we were only able to demonstrate a reduction in the 1205 tumor growth by about 20% (unpublished data).

It can therefore be stressed that this combination of LMW-PEI and unmodified RNA ribozymes is a new and extremely effective agent for the selective reduction of the expression of genes in vivo in animal tests.

### DESCRIPTION OF THE DRAWINGS

FIG. 1 FGF-BP RNA (800 nucleotides)—degradation in 20 ng/ml (RNase A (plus/minus LMW polyethylene imine (PEI)—complexing):

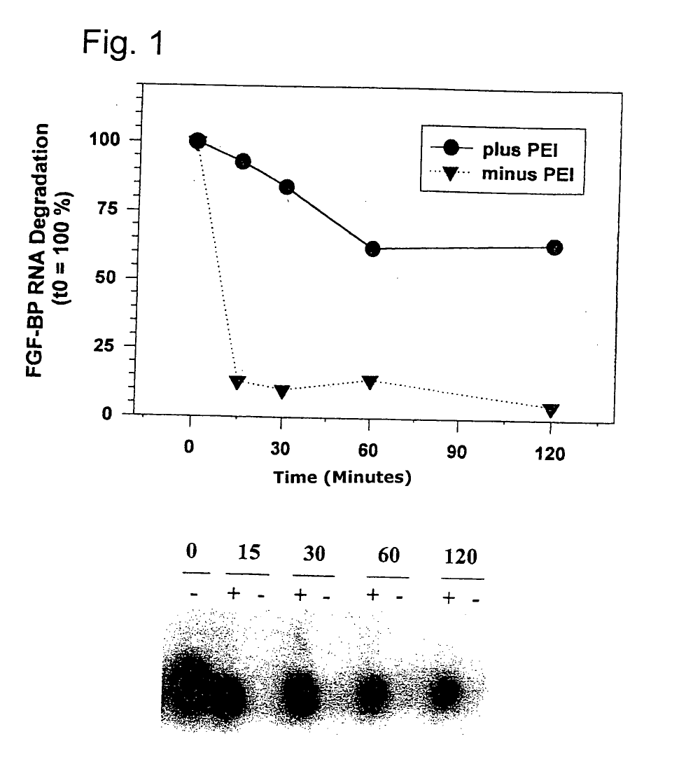

While in the incubation with RNase A (final concentration 20 ng/ml) as expected the non-complexed RNA is almost completely decomposed after 15 minutes, more than 60% of the molecules are still intact from the PEI-complexed RNA even after 2 hours.

FIG. 2 FGF-BP RNA (800 nucleotides)—degradation in 1% FCS (plus/minus LMW polyethylene imine (PEI)—complexing):

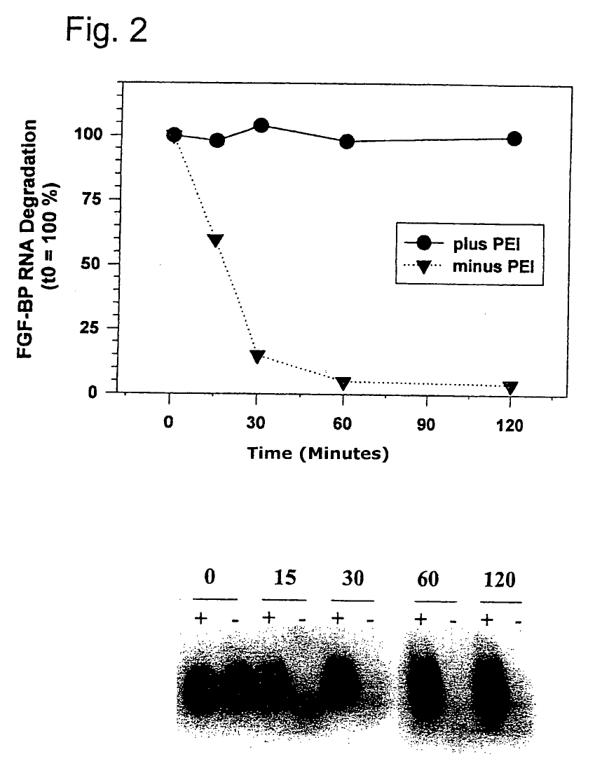

A very similar kinetics as in FIG. 1 also occurs after incubation with fetal calf serum (FIG. 2). Here the non-complexed RNA is completely decomposed after about 30 minutes after incubation with 1% fetal calf serum while the PEI-complexed RNA is still completely intact even after 120 minutes. Even upon an increase in the concentration to 40% fetal calf serum the PEI-complexed RNA is markedly more stable in comparison with the non-complexed RNA. The results were identical for the RNA molecules of different lengths. The data of the graphs were averaged from two independent experiments.

FIG. 3 FGF-BP ribozymes complexed with low-molecular PEI inhibit FGF-BP mRNA expression in ME-180 platelet epithelium carcinoma cells after transient transfection.

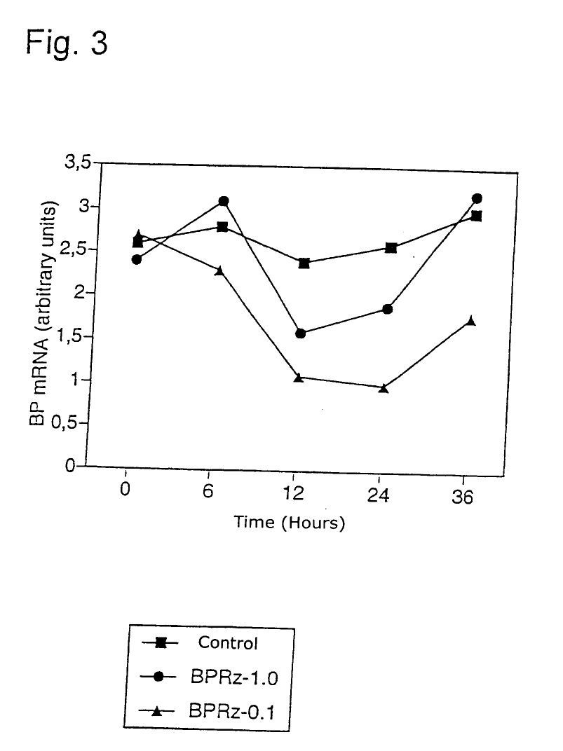

FIG. 4 FGF-BP ribozymes complexed with low-molecular PEI (N/P ratio 1:13) inhibit FGF-BP mRNA expression in ME-180 platelet epithelium carcinoma cells after transient transfection.

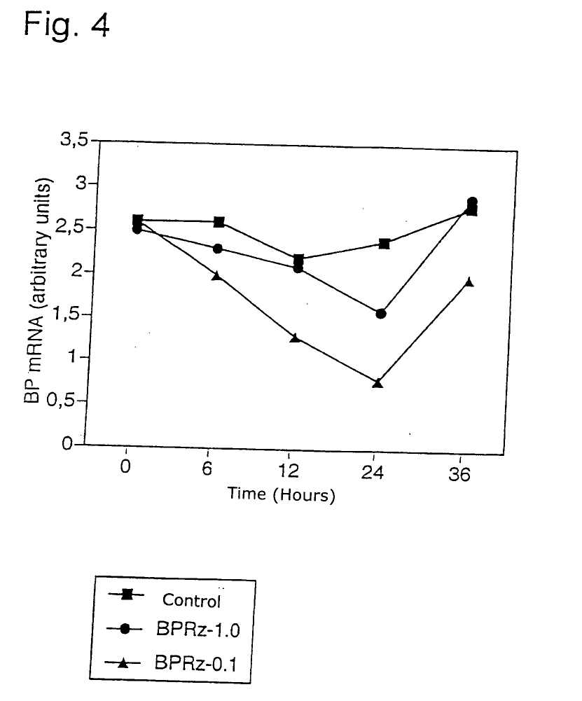

FIG. 5 FGF-BP ribozymes complexed with low-molecular PEI (N/P ratio 1:53) inhibit FGF-BP mRNA expression in ME-180 platelet epithelium carcinoma cells after transient transfection.

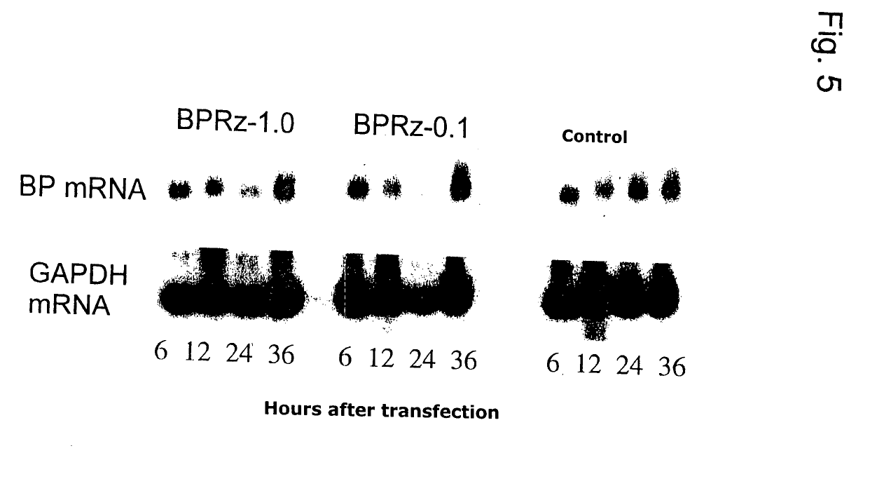

As can be seen from FIGS. 3-5 transfection with LMW-PEI ribozymes results in very great ribozyme-specific reduction in FGF-BP gene expression.

The maximum reduction was about 70% compared to the control cells. Both ribozyme-PEI ratios of 1:13 and 1:53 exhibited similar effectiveness. Interestingly the low ribozyme dose of 0.1 μg was somewhat more effective in both PEI ratios so that it can be assumed that even still lower doses can already be highly efficient.

FIG. 6 pleiotrophin (PTN) ribozymes complexed with low-molecular PEI (N/P ratio 1:66.66) inhibit the growth of human melanoma tumor cell transplants (1205 cells) in athymic nude mice after systemic application.

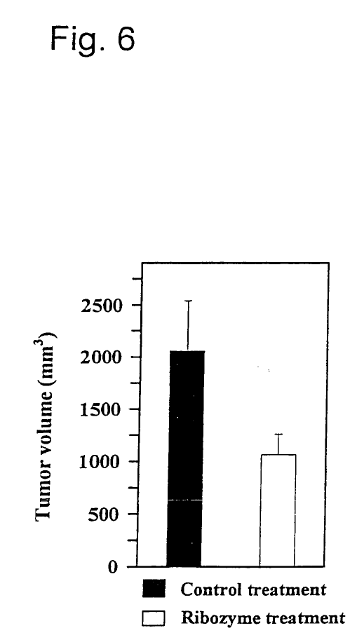

1205 tumor cells (1×10⁶ cells/injection) were subcutaneously injected into the flanks of athymic nude mice and treated either with PTN-ribozymes complexed with LMW-PEI (8 μg of ribozyme/injection) intraperitoneally (i.p.) at an interval of 2 days (treatment group=8 animals) or only with LMW-PEI at the same interval (control group=7 animals). After 20 days a specific ribozyme-afforded inhibition of the tumor growth by about 50% was exhibited.

1. A method of using complexes of RNA molecules with polyethylene imines (PEI) which are optionally modified with hydrophilic polymers coupled thereto for protecting RNA from enzymatic or non-enzymatic, intra- or extracellular decomposition.

2. A method as set forth in claim 1 in which the RNA molecules are protected from intra- or extracellular decomposition by nucleases.

3. A method as set forth in one of claims 1 or 2 characterised in that the RNA are liberated in active form after the incorporation thereof into the cell intracellularly from the complex and can exert biological functions.

4. A method as set forth in one of claims 1 through 3 in which the RNA is a messenger RNA (mRNA), a ribosomal RNA (rRNA), a transfer RNA (tRNA), a nuclear RNA, a RNA ribozyme, a RNA aptamer, an in vitro transcribed RNA or a chemically synthesised RNA, wherein all said RNA molecules can be chemically modified.

5. A method as set forth in one of claims 1 through 4 in which the RNA entirely or partly codes for a therapeutically effective peptide or protein or for a cellular suicide gene.

6. A method as set forth in one of claims 1 through 5 characterised in that the RNA has between 10 and 10,000 nucleotides, preferably between 15 and 1500 nucleotides, in particular between 20 and 1000 nucleotides.

7. A method as set forth in one of claims 1 through 4 and 6 characterised in that the RNA is a ribozyme, preferably a hammerhead ribozyme.

8. A method as set forth in one of claims 1 through 7 characterised in that the PEI is not modified with hydrophilic polymers coupled thereto.

9. A method as set forth in one of claims 1 through 8 characterised in that the PEI is modified with one or more hydrophilic polymers coupled thereto.

11. A method as set forth in one of claims 1 through 9 characterised in that the hydrophilic polymer is modified with one or more PEI coupled thereto.

11. A method as set forth in one of claims 1 through 7, 9 and 10 characterised in that the hydrophilic polymer is polyethylene glycol (PEG).

12. A method as set forth in one of claims 1 through 11 characterised in that the PEI has a molecular weight of between 700 and 1,000,000 Da, preferably between 1400 and 25,000 Da, in particular between 1600 and 15,000 Da.

13. A method as set forth in one of claims 1 through 12 in which the molar ratio of the nitrogen atoms in the PEI to the phosphorus atoms in the RNA (N/P value) is between 1 and 100, preferably between 2 and 40, in particular between 3 and 10.

14. A method as set forth in one of claims 1 through 13 in which the PEI is modified by a cellular ligand.

15. A method as set forth in one of claims 1 through 14 characterised in that the RNA concentration is 0.01-1000 μg/ml, preferably 1-100 μg/ml, particularly preferably 10-40 μg/ml.

16. A complex comprising a ribozyme of a chain length of 20-600 nucleotides, preferably a hammerhead ribozyme, and polyethylene imine (PEI) which is optionally modified with hydrophilic polymers coupled thereto.

17. A complex as set forth in claim 16 in which the PEI is not modified with hydrophilic polymers coupled thereto.

18. A complex as set forth in claim 16 in which the PEI is modified with one or more hydrophilic polymers coupled thereto.

19. A complex as set forth in claim 16 in which the hydrophilic polymer is modified with one or more PEI coupled thereto.

20. A complex as set forth in one of claims 16, 18 and 19 in which the hydrophilic polymer is polyethylene glycol (PEG).

21. A complex as set forth in one of claims 16 through 20 in which the PEI has a molecular weight of between 700 and 1,000,000 Da, preferably between 1400 and 25,000 Da, in particular between 1600 and 15,000 Da.

22. A complex as set forth in one of claims 16 through 21 in which the molar ratio of the nitrogen atoms in the PEI to the phosphorus atoms in the ribozyme (N/P value) is between 1 and 100, preferably between 2 and 40, in particular between 3 and 10.

23. A complex as set forth in one of claims 16 through 22 in which the PEI is modified by a cellular ligand.

24. A method of producing a complex as set forth in one of claims 16 through 23 characterised in that the ribozyme is complexed with the PEI optionally modified by a cellular ligand or optionally by a further polymer, by mixing of the dilute solutions.

25. A method as set forth in claim 24 characterised in that the ribozyme concentration is 0.01-1000 μg/ml, preferably 1-100 μg/ml, particularly preferably 10-40 μg/ml.

26. A pharmaceutical composition containing an effective amount of at least one complex as set forth in one of claims 16 through 23 and one or more physiologically harmless carriers.

27. A composition as set forth in claim 26 in which the ribozyme is present in any concentration which is sub-toxic for cells or for the respective organism or which is still not permanently cell-damaging at least in treatment over several hours.

28. Use of a composition as set forth in claim 26 or claim 27 for gene therapy.

29. Use of a complex as set forth in one of claims 16 through 23 for protecting ribozymes from enzymatic or non-enzymatic decomposition upon storage or upon transportation of the ribozymes, modified or unmodified, in aqueous solutions, or in the use of ribozymes, modified or unmodified, in laboratory experiments.

30. Use of a complex as set forth in one of claims 16 through 23 for the incorporation of ribozymes into cells, preferably into eucaryontic cells and for the intracellular liberation thereof in biologically active form.
